(19) United States
(12) Patent Application Publication
(10) Pub. No.: US 2016/0015385 A1
(43) Pub. Date: Jan. 21, 2016

(54) PUNCTURE APPARATUS

(71) Applicant: TERUMO MEDICAL INNOVATIONS, Mountain View, CA (US)

(72) Inventors: Masakatsu KAWAURA, Sunnyvale, CA (US); Nao YOKOI, Sunnyvale, CA (US); Yuusuke TAKAHASHI, Hadano-shi (JP)

(73) Assignee: TERUMO MEDICAL INNOVATIONS, Mountain View, CA (US)

(21) Appl. No.: 14/802,394

(22) Filed: Jul. 17, 2015

Related U.S. Application Data
(60) Provisional application No. 62/026,302, filed on Jul. 18, 2014.

(51) Int. Cl. A61B 17/06; A61B 17/34; A61F 2/00

(52) U.S. Cl. CPC A61B 17/06109; A61F 2/0045; A61B 17/3468; A61B 2017/061; A61B 2017/06104; A61B 2017/00805

(57) ABSTRACT

A puncture assembly is disclosed, which includes a sheath and an insertion portion. The sheath includes a main body to guide a sling, and a puncture portion disposed at a distal end of the main body, the puncture portion including a position-control portion at the distal end of the main body and a dissecting portion, wherein the puncture portion is configured to rotate in an extended curved line of the main body together with the main body to create a through hole from an outside a living body through obturator foramens of the living body. The insertion portion is configured to be inserted into a hollow lumen of the sheath and has an arcuate shape corresponding to a shape of the sheath.

### CROSS REFERENCES TO RELATED APPLICATIONS

This application claims priority under 35 U.S.C. 119 to U.S. Provisional Application No. 62/026,302 filed on Jul. 18, 2014, which is incorporated herein by reference in its entirety.

### TECHNOLOGICAL FIELD

The present disclosure relates to a puncture apparatus and a puncturing method.

### BACKGROUND

In a patient suffering from urinary incontinence, particularly stress urinary incontinence, urine leakage (involuntary urination) can occur due to an abnormal pressure exerted, for example, during a normal exercise or by laughing, coughing, or sneezing. This can be attributable, for example, to loosening of a pelvic floor muscle, which is a muscle supporting a urethra, caused by childbirth.

For treatment of urinary incontinence, surgical therapy can be effective. For example, a tape-shaped implant called “sling, or mesh” can be placed indwelling in the body to support the urethra by the sling (refer to, for example, Japanese Patent Laid-Open No. 2010-99499). In order to put a sling indwelling in the body, an operator incises a vagina with a surgical knife, dissects a biological tissue (living body tissue) between the urethra and the vagina, and provides communication between the dissected biological tissue site and outside the body through an obturator foramen by using a puncture needle or the like which is curved in an arcuate shape (this operation will hereinafter be referred to as “puncturing operation”). Then, in such a state, the sling is placed indwelling in the body.

However, this procedure depends mainly on the operator's skill.

It is hard to accurately position the sling under the mid-urethra position and middle positions between urethra and vagina wall since an operator decides the position and incises the vagina simply by palpation or visual contact.

Since the sling (mesh) is delivered through a narrow path of a needle in a region between an obturator foramen and groin (pelvic) skin, the sling might be curled when it is delivered through the path to the area. Then, the fixing force of the sling may be reduced because the sling-contacted area (a surface area of the sling) in the tissues gets smaller.

### SUMMARY

In accordance with an exemplary embodiment, a puncture apparatus and a puncturing method are disclosed by which a biological tissue can be punctured accurately and satisfactorily.

In accordance with an exemplary embodiment, a sheath is disclosed comprising: a main body to guide a sling; and a puncture portion disposed at the distal end of the main body comprising a position-control portion at the distal end and a dissecting portion.

In accordance with an exemplary embodiment, a urethra-insertion member insertable into the urethra is disclosed comprising: an elongated urethra-insertion portion; a chamber disposed at the proximal end of the elongated urethra-insertion portion, and connected so as to enclose at least a portion of the elongated urethra-insertion portion; and a hub capable of sliding relatively to the chamber in the longitudinal direction of the elongated urethra-insertion portion, and enclose at least a portion of the chamber.

In accordance with an exemplary embodiment, a method for forming a through hole passing the living tissue between the urethra and the vagina by puncturing a living tissue is disclosed, comprising: inserting a urethra-insertion member in the urethra in a subject, measuring a length of urethra in a subject, and changing the length of the urethra-insertion member so as to form a through hole passing the living tissue under the mid-urethra adjacent the center of the length of urethra, connecting a puncture needle to the urethra-insertion member, after changing.

In accordance with an exemplary embodiment, a puncture assembly is disclosed comprising: a main body to guide a sling; and a puncture portion disposed at a distal end of the main body, the puncture portion including a position-control portion at the distal end of the main body and a dissecting portion.

In accordance with an exemplary embodiment, a puncture assembly is disclosed, comprising: a sheath, the sheath including a main body to guide a sling, and a puncture portion disposed at a distal end of the main body, the puncture portion including a position-control portion at the distal end of the main body and a dissecting portion, wherein the puncture portion is configured to rotate in an extended curved line of the main body together with the main body to create a through hole from an outside a living body through obturator foramens of the living body; and an insertion portion configured to be inserted into a hollow lumen of the sheath, the insertion portion having an arcuate shape corresponding to a shape of the sheath.

In accordance with an exemplary embodiment, a method is disclosed of forming a path in living body tissue, the method comprising: inserting an insertion member into a living body, the insertion member comprising at least one of a urethral-insertion portion that is inserted into a urethra and has an elongated shape and a vaginal-insertion portion that is inserted into a vagina and has an elongated shape; inserting a puncture assembly into the living body, the puncture assembly comprising a sheath having a main body to guide a sling, and a puncture portion disposed at a distal end of the main body, the puncture portion including a position-control portion at the distal end of the main body and a dissecting portion, wherein the puncture portion is configured to rotate in an extended curved line of the main body together with the main body to create a through hole in a biological tissue from an outside a living body through obturator foramens of the living body; and detecting a positional relationship of the distal end of the main body of the puncture assembly with the position-control portion of the sheath; and determining a puncture route of the puncture portion of the puncture assembly.

### BRIEF DESCRIPTION OF THE DRAWINGS

FIG. 1 is a perspective view showing an exemplary first embodiment of a puncture apparatus according to the present disclosure.

FIG. 2 is a side view of the puncture apparatus shown in FIG. 1.

FIG. 3A is a plan view showing a fixing portion of a frame possessed by the puncture apparatus shown in FIG. 1.

FIG. 3B is a side view showing a fixing portion of a frame possessed by the puncture apparatus shown in FIG. 1.

FIG. 4A is a side view of an insertion instrument possessed by the puncture apparatus shown in FIG. 1.

FIG. 4B is a partial magnified view of insertion member possessed by the puncture apparatus shown in FIG. 4A.

FIG. 4C is a partial magnified view of insertion member possessed by the puncture apparatus shown in FIG. 4A.

FIG. 4D is a sectional view taken along line 4D-4D in FIG. 4C.

FIG. 4E is a sectional view taken along line 4E-4E in FIG. 4C.

FIG. 5A is a side view illustrating a positional relationship between a puncture member and obturator foramens (pelvis).

FIG. 5B is a front view illustrating a positional relationship between a puncture member and obturator foramens (pelvis).

FIG. 6 is a partial magnified view of a vaginal insertion member possessed by the insertion instrument shown in FIG. 4A.

FIG. 7A is a sectional view showing an example of a shape of a vaginal wall.

FIG. 7B is a sectional view showing a state in which a vaginal insertion portion has been inserted in an inside of the vagina shown in FIG. 7A.

FIG. 8A is a perspective view of a puncture member possessed by the puncture apparatus shown in FIG. 1.

FIG. 8B is a sectional view taken along line 8B-8B in FIG. 8A.

FIG. 8C is a sectional view shown in FIG. 8A.

FIG. 8D is a partial sectional view of the puncture member shown in FIG. 8A.

FIG. 9A is a view illustrating an operation procedure of the puncture apparatus shown in FIG. 1.

FIG. 9B is another view illustrating an operation procedure of the puncture apparatus shown in FIG. 1

FIG. 10 is a view illustrating the operation procedure of the puncture apparatus shown in FIG. 1.

FIG. 11 is a side view illustrating a relationship between the puncture apparatus and the pelvis in the state shown in FIG. 10.

FIG. 12 is operation member of the puncture apparatus shown in FIG. 8A.

FIG. 13 is a perspective view showing an implant according to the first exemplary embodiment of the present disclosure.

### DETAILED DESCRIPTION

In accordance with an exemplary embodiment, a puncture apparatus and a puncturing method according to the present disclosure will be described in detail below, referring to exemplary embodiments thereof illustrated in the accompanying drawings.

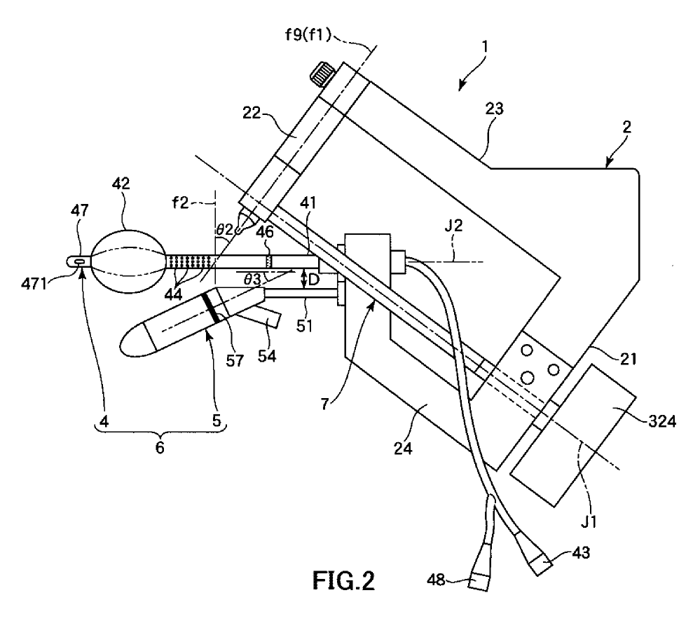

In the following, for convenience of description, a left side in FIG. 2 will be referred to as “distal (side),” a right side as “proximal (side),” an upper side as “upper (side),” and a lower side as “lower (side).” In addition, FIG. 2 shows the puncture apparatus in a state of being yet to be used, which state will hereinafter be referred to also as “initial state,” for convenience of description. Further, a state wherein the puncture apparatus (insertion instrument) shown in FIG. 2 is mounted on a patient will be referred to also as “mounted state.”

Puncture Apparatus

In accordance with an exemplary embodiment, the puncture apparatus 1 is an apparatus to be used for treatment of female urinary incontinence, for example, to be used in a process in which a biological tissue-supporting implant for treatment of urinary incontinence is implanted into a living body.

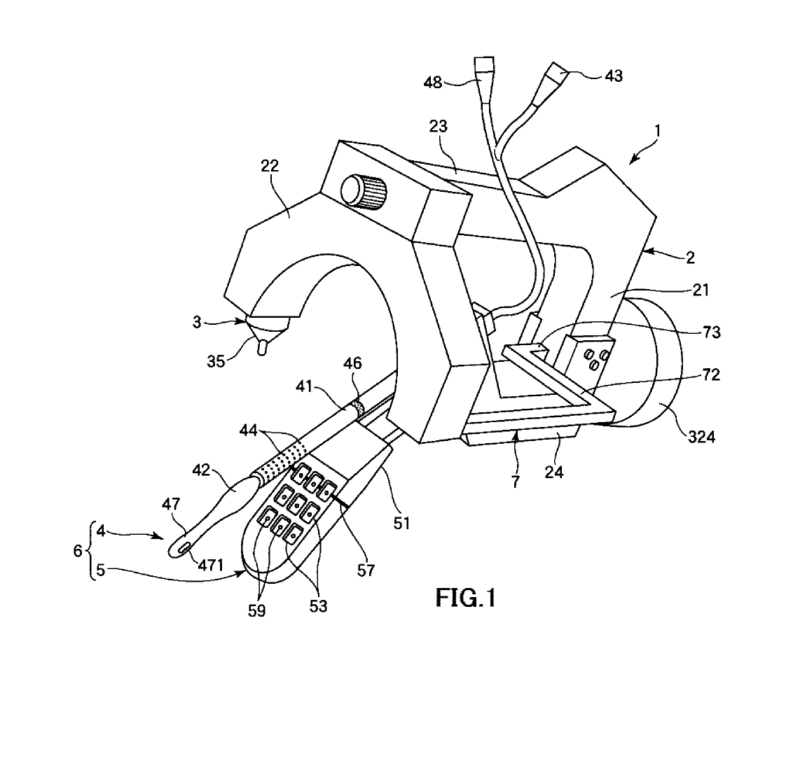

As shown in FIGS. 1 and 2, the puncture apparatus 1 can include a frame (support unit) 2, a puncture member 3, a urethral insertion member 4, and a vaginal insertion member 5. The puncture member 3, the urethral insertion member 4, and the vaginal insertion member 5 can be supported on the frame 2. In the puncture apparatus 1, for example, the urethral insertion member 4 and the vaginal insertion member 5 constitute an insertion instrument 6.

Frame

As shown in FIGS. 1 and 2, the frame 2 can include a grasping portion 21 to be grasped in the mounted state, a guide portion (holding portion) 22 guiding the puncture member 3, an interlock portion 23 interlocking the grasping portion 21 and the guide portion 22 to each other, and a fixing portion 24 to which the insertion instrument 6 is fixed.

The grasping portion 21 can be located on a proximal end in the puncture apparatus 1, and extends in a direction substantially orthogonal to an axis J1. It is to be noted that “axis J1” refers to an axis which serves as a center (axis) of turn when the puncture member 3 is turned.

The guide portion 22 is located on a distal end in the puncture apparatus 1, and is disposed opposite to the grasping portion 21. The guide portion 22 is arch-shaped.

The interlock portion 23 is interlocking the grasping portion 21 and the guide portion 22 to each other. In addition, the interlock portion 23 is in a form of a bar extending substantially in parallel to the axis J1. The interlock portion 23 can function also as a grasping portion, like the grasping portion 21, to allow an operator to use the puncture apparatus 1 by grasping the interlock portion 23.

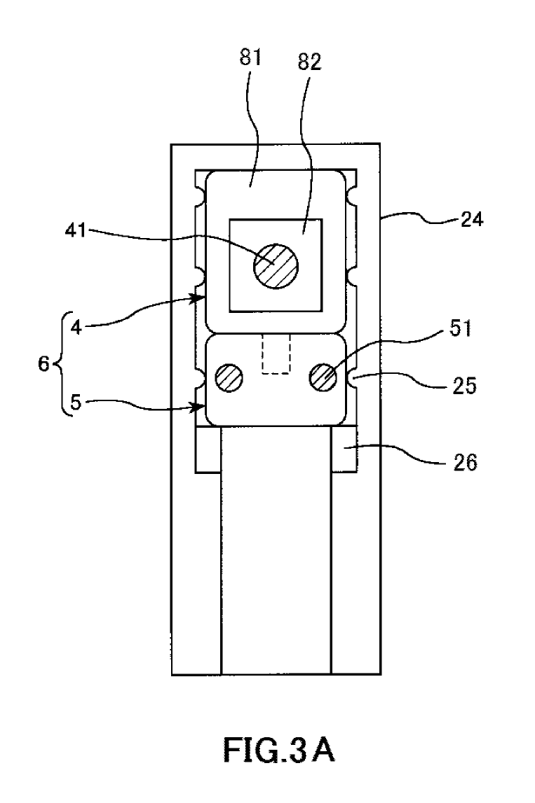

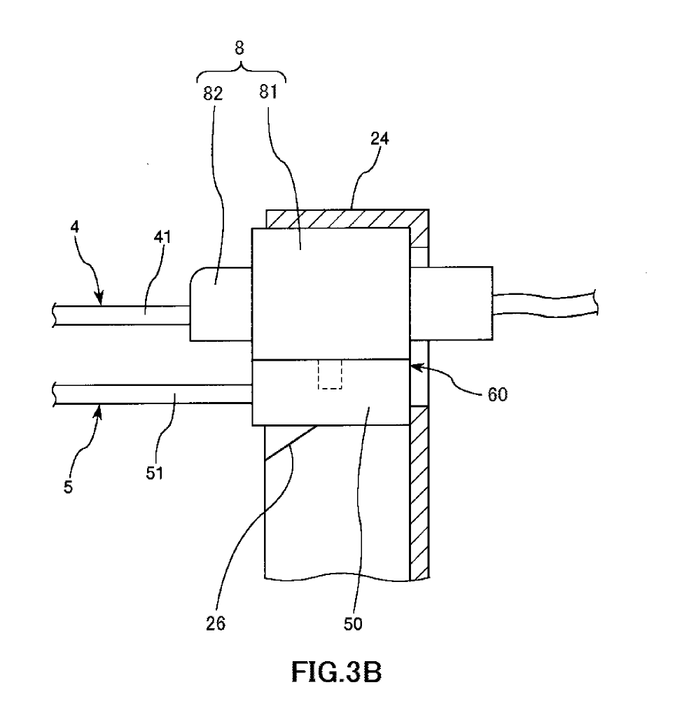

As shown in FIGS. 3A and 3B, the fixing portion 24 is provided with a recess 243 in which to fit a support part 60 (described later) of the insertion instrument 6. With the support part 60 fitted in the recess 243, the insertion instrument 6 can be fixed to the fixing portion 24. A lock portion (not shown) can fix the insertion instrument 6 to fixing portion 24.

The fixing portion 24 has a rib structure 25 on the inner wall of the fixing portion 24. The rib structure 25 protrudes from the inner wall of the fixing portion 24, and decreases the friction between the inner wall and the support part 60. The fixing portion 24 has a locking part, which can control the insertion of the insertion instrument 6 into the fixing portion 24. The fixing portion 24 has a guide member 26 to help the support part 60 inserted into inside the fixing portion 24. The guide member 26 can be a slot made on the inner wall of the fixing portion 24, and has a narrower thickness of the wall of the fixing portion 24. The guide member 26 can have a widened entrance for the support part 60 to be inserted at the edge of the fixing portion 24 to ease the insertion of the fixing portion 24 into the slot.

Puncture Apparatus

Puncture Member

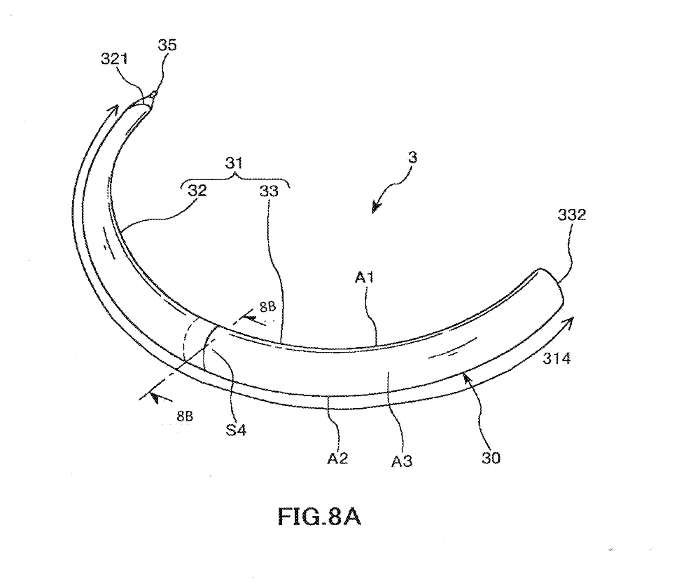

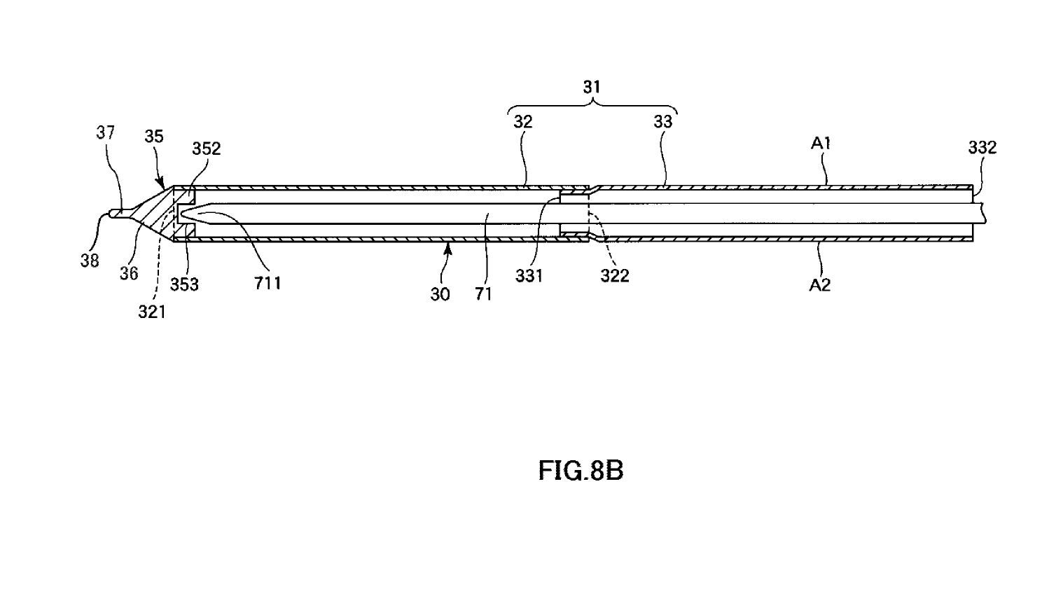

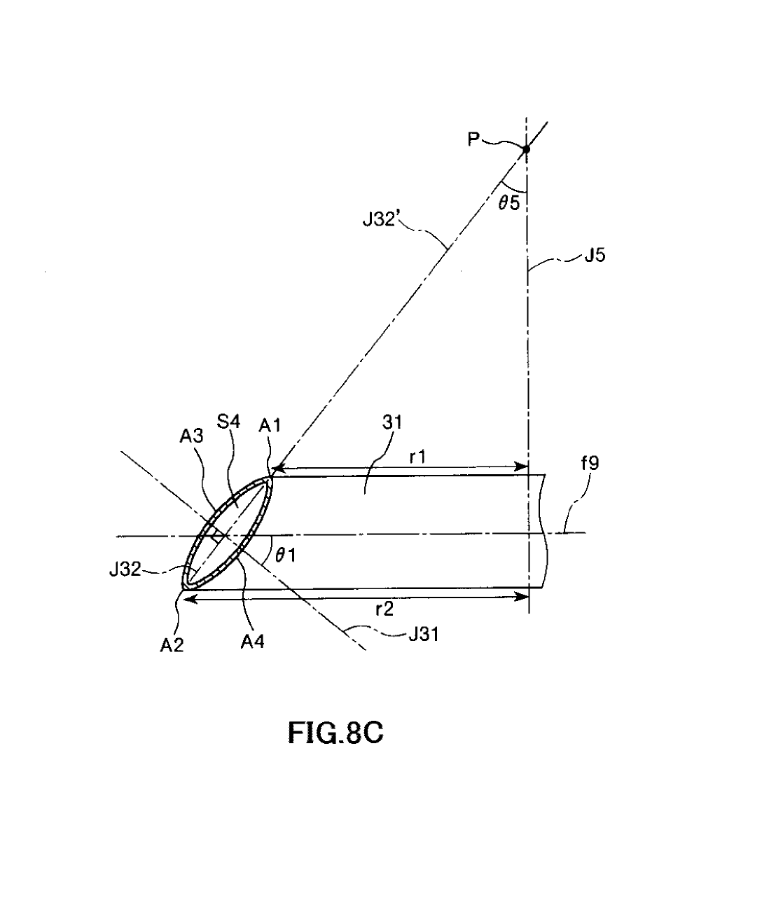

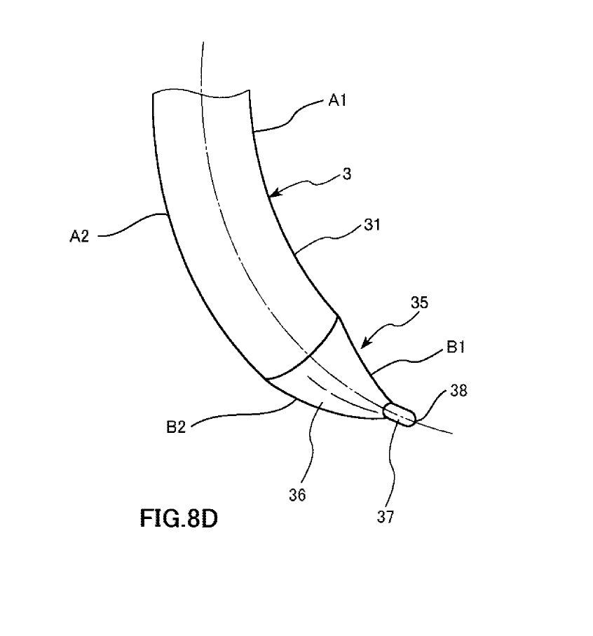

The puncture member 3 is puncturing a living body, while securing a target puncture route R, to form a puncture hole. As shown in FIG. 8B, the puncture member 3 includes a sheath 30, a puncture portion 35 provided at a distal end of the sheath 30, and an operating member 7 (insertion portion 71) operating the sheath 30. The sheath 30 and the puncture portion 35 constitute a puncture needle. In addition, the sheath 30 has a tube-shaped main body 31.

Operating Member

The operating member 7 is a member operating the sheath 30. Such an operating member 7, as shown in FIG. 1, can include the insertion portion 71, a shaft portion 73, and an interlock portion 72 interlocking the insertion portion 71 and the shaft portion 73 to each other. The insertion portion 71, the interlock portion 72, and the shaft portion 73 can be formed integrally, or, alternatively, at least one of the portions may be formed as a separate body in relation to the other portions.

The insertion portion 71 is a portion to be inserted in the sheath 30, and functions as a stylet that supports the sheath 30 from the inside. With the insertion portion 71 inserted in the sheath 30, the sheath 30 is interlocked to the operating member 7, whereby it is enabled to turn the sheath 30 by the operating member 7. Such an insertion portion 71 is in an arcuate shape corresponding to the shape of the sheath 30. A center angle of the insertion portion 71 is set in conformity with a center angle of the sheath 30. For example, a distal portion 711 of the insertion portion 71 can be tapered. The presence of the tapered distal portion 711 can enable relatively smooth insertion of the insertion portion 71 into the sheath 30.

The shaft portion 73 is a turning shaft, which intersects a center 0 of the insertion portion 71 and serves as a center of turn of the sheath 30 (puncture portion 35). The shaft portion 73 extends along an axis J1 orthogonal to a plane f1 that contains the insertion portion 71.

The interlock portion 72 interlocks a proximal portion of the insertion portion 71 and a distal portion of the shaft portion 73 to each other. In addition, the interlock portion 72 can be substantially L-shaped, with a substantially rectangular bend at an intermediate portion of the interlock portion 72. The interlock portion 72 can function also as a grasping portion to be grasped by an operator at the time of operating the operating member 7.

Such an operating member 7 can be configured to be higher than the sheath 30 in rigidity. The material constituting the operating member 7 is not specifically restricted. Examples of the material applicable here can include various metallic materials such as stainless steels, aluminum, aluminum alloys, titanium, and titanium alloys.

Sheath

The sheath 30 can include a tube-shaped main body 31, and the puncture portion 35 provided at a proximal portion of the main body 31.

As shown in FIGS. 8A and 8C, in an exemplary embodiment, a main body 31 of the sheath 30 can be curved in a bow-like shape, can be rigid, and can be composed of two split pieces so that it can be split at an intermediate portion of the main body 31. For example, the main body 31 can be divided into a distal split piece 32 and a proximal split piece 33. The distal split piece 32 and the proximal split piece 33 approximately equal in length, and the boundary between the distal split piece 32 and the proximal split piece 33 is located at a central portion S4.

As shown in FIG. 8A, the distal split piece 32 can be tubular in shape, and has a distal end opening 321 and a proximal end opening 322. Similarly, the proximal split piece 33 can be tubular in shape, and has a distal end opening 331 and a proximal end opening 332. A distal portion of the proximal split piece 33 can be inserted into a proximal portion of the distal split piece 32, whereby the distal split piece 32 and the proximal split piece 33 connected to each other. With the proximal split piece 33 thus inserted into the distal split piece 32, a step which can be generated at a boundary between the split pieces 32 and 33 is insusceptible to be caught on a biological tissue, so that puncturing of a living body by the puncture member 3 can be performed smoothly. It is to be noted that on the contrary to this exemplary embodiment, the distal split piece 32 may be inserted into the proximal split piece 33 to connect the split pieces 32 and 33 to each other.

The material constituting the main body 31 is, for example, a rigid material that can maintain the shape and an internal space of the main body 31 in the state of being inserted in a body. Examples of such rigid material applicable here can include various resin materials such as polyethylene, polyimides, polyamides, polyester elastomers, polypropylene, etc. and various metallic materials such as stainless steels, aluminum, aluminum alloys, titanium, and titanium alloys. It is to be noted that instead of adopting a rigid material constituting the main body 31, other material than rigid material may also be adopted, in which case a wall of the main body 31 is reinforced with a reinforcement member, whereby the preferable properties as above-mentioned are attained. For example, a high-strength braiding may be embedded in the wall of the main body 31, whereby the shape and the internal space of the main body 31, in the state of being inserted in a body, can be maintained. Another example of the reinforcement member is a spiral body, which may be embedded in the wall of the main body 31, whereby the main body 31 can have flexibility while retaining the internal space to such an extent that an inserted body can be slid therein.

The main body 31 as above is provided with the puncture portion 35 at the distal end of the main body 31. As shown in FIG. 8D, the puncture portion 35 has a dissecting portion 36, which is tapered off, and a proximal portion 352 provided on the proximal end of the dissecting portion 36. The proximal portion 352 can be inserted in the main body 31, whereby the puncture portion 35 is held inside the main body 31 in a freely detachable manner. In accordance with an exemplary embodiment, the proximal portion 352 can be fitted in the main body 31 with such a force that unintended detachment of the puncture portion 35 from the main body 31 can be relatively prevented. Further, the puncture portion 35 may be configured integrally with the main body 31.

In addition, the proximal portion 352 can be provided with an engaging portion 353 for engagement with the distal portion 711 of the insertion portion 71. The engaging portion 353 can be composed essentially of a recess. In an inserted state wherein the puncture member 3 is inserted in the insertion portion 71, the distal portion 711 is located inside the engaging portion 353. The engaging portion 353 thus provided can help ensure that displacement of the puncture portion 35 relative to the insertion portion 71 is suppressed, and puncturing of a living body by the puncture member 3 can be carried out relatively smoothly.

In accordance with an exemplary embodiment, in the case where at least the distal portion 711, of the insertion portion 71, is flat-shaped in cross section, the cross-sectional shape of the engaging portion 353 can be set in conformity with the cross-sectional shape of the distal portion 711, for example, the engaging portion 353 can also be flat-shaped in cross section, which can help ensure that when the engaging portion 353 the distal portion 711 are in engagement with each other, the flat shape of the engaging portion 353 the flat shape of the distal portion 711 overlap each other. This overlap can help ensure that the sheath 30 is restrained from rotating relative to the insertion portion 71, about the axis thereof, for example, about an axis extending along the puncturing direction. Consequently, a curved puncture route, which can reliably pass between the urethra 1300 and the vagina 1400 can be relatively secured.

The material constituting the puncture portion 35 is not specifically restricted. For example, the same materials as those for the main body 31 can be used for the puncture portion 35.

As shown in FIG. 8A, the main body 31 can have a curved portion 314 ranging at least to a predetermined distance from the distal end of the main body 31. The curved portion 314 is a portion, which has been so shaped that it will be in an arc-shaped (arcuate) curved form in a natural state with no external force exerted thereon.

In accordance with an exemplary embodiment, a center angle of the curved portion 314 is not particularly limited, and can be set, as necessary, according to various conditions. In this case, as will be described later, the center angle can be set that the curved portion 314 can enter a patient body via an inguinal region on one side of the patient together with the puncture needle 11 and can be moved past a region between a urethra 1300 and a vagina 1400, to reach a vicinity of an inguinal region on the other side. For example, this center angle, for example, can be in the range of 150 to 270 degrees, for example, 170 to 250 degrees, and for example, preferably 190 to 230 degrees.

In addition, the main body 31 can be configured that at least its curved portion 314 is elastic. As shown in FIG. 8C, the main body 31 is flat-shaped in cross section. For example, the cross-sectional shape of the curved portion 314 at a central portion S4 in the longitudinal direction thereof is a flat shape, which can include a minor axis J31 and a major axis J32. As will be described later, an implant main body 91 can be disposed inside the main body 31, in place of the puncture needle 11. In this case, with the main body 31 flat-shaped, a posture of the implant main body 91 inside the main body 31 can be controlled.

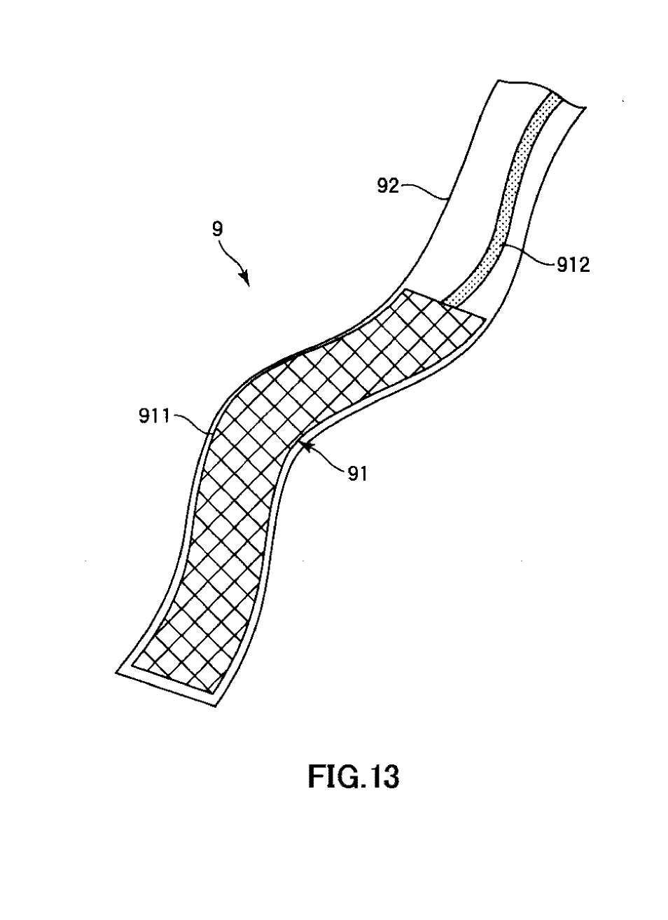

In accordance with an exemplary embodiment, a width (a length in a direction of the major axis J32) of an internal space of the main body 31 is designed to be approximately equal to a width of a main body portion 911 (described later) of the implant main body 91 (see FIG. 13), which can help ensure that even when the implant main body 91 is moved, a frictional resistance between the implant main body 91 and the internal space of the main body 31 is lowered, and no unnecessary force is applied to the implant main body 91, so that the main body portion 911 can be disposed in a sufficiently expanded (spread) state in the inside of the main body 31. In accordance with an exemplary embodiment, the width (the length in the direction of the major axis J32) of the internal space of the main body 31 may be shorter than the width of the main body portion 911, which can help ensure that the width of the main body 31 is set smaller, so that a less invasive puncture member 3 can be realized.

In accordance with an exemplary embodiment, the flat shape of the main body 31 is not specifically restricted. Examples of the flat shape applicable here can include ellipses, sectionally convexed lens-like shapes, rounded-cornered rhombuses, rounded-cornered rectangles (flat shapes), and spindle-like shapes enlarged (enlarged in diameter) at a central portion as compared with both end portions of the main body 31.

Hereinafter, for convenience of description, as shown in FIG. 8C, an end portion located on an inner side (one end portion) in the direction of the major axis J32 will be referred to also as “inner circumferential portion A1,” an end portion located on an outer side (other end portion) will be referred to also as “outer circumferential portion A2,” a surface oriented upward will be referred to also as “front surface A3,” and a surface oriented downward will be referred to also as “back surface A4.”

When a plane containing both the center of the arc of the central portion S4 and the center of the cross-sectional shape relative to the longitudinal direction of the main body 31 (a plane containing the center axis of the main body 31) is referred to as plane f9 and an angle formed between the plane f9 and the minor axis J31 at the central portion S4 is referred to as inclination angle θ1, the inclination angle θ1 is preferably an acute angle. With the inclination angle θ1 set to be an acute angle, the implant 9 (described later) can be disposed substantially in parallel to the urethra (urethral lumen) 1300, whereby the urethra 1300 can be supported relatively more effectively.

In accordance with an exemplary embodiment, the inclination angle θ1 is not particularly limited, insofar as it is an acute angle. For example, the inclination angle θ1 can be about 20 to 60 degrees, more preferably about 30 to 45 degrees, and further preferably about 35 to 40 degrees, which can help ensure that the above-mentioned effects can be further enhanced.

In accordance with an exemplary embodiment, for example, for the inclination angle θ1 to satisfy the above-mentioned numerical range throughout a whole region in an extending direction of the curved portion 314. However, the above-mentioned effect can be displayed if only the inclination angle θ1 satisfies the above-mentioned numerical range at least in the central portion S4 in the extending direction of the curved portion 314. In accordance with an exemplary embodiment, the above-mentioned “central portion S4” refers to a region that includes a part located between the urethra 1300 and the vagina (vaginal lumen) 1400, at least, in a state wherein the puncture member 3 is puncturing a living body (a state wherein the main body 31 is disposed in a living body).

In accordance with an exemplary embodiment, the configuration of the main body 31 is shown in FIG. 8C, wherein the main body 31 is so formed that the major axis J32 is inclined against a center axis J5 of the arc and that the center axis J5 of the arc and an extension line J32′ of the major axis J32 have an intersection P. In this case, an angle θ5 formed between the center axis J5 and the extension line J32′ can be equal to the inclination angle θ1.

In accordance with an exemplary embodiment, as shown in FIG. 8B, the main body 31 has the inner circumferential portion A1 located at an inner circumferential edge (in plan view as viewed from the direction of the center axis J5 of the main body 31) and having a minimum radius of curvature, r1, and also has the outer circumferential portion A2 located at an outer circumferential edge (in the plan view) and having a maximum radius of curvature, r2, and that the inner circumferential portion A1 and the outer circumferential portion A2 are located to be spaced (deviated) from each other along the direction of the center axis J5.

As above-mentioned, the main body 31 can be flat-shaped and can be insusceptible to collapse in the longitudinal direction thereof, so that the spacing between the inner circumferential portion A1 and the outer circumferential portion A2 will hardly be varied. In addition, the inner circumferential portion A1 and the outer circumferential portion A2 can be greater in curvature and can be less susceptible to deformation, as compared with the front surface A3 and the back surface A4.

In accordance with an exemplary embodiment, the sheath 30 can be formed of a material that is elastic and that can maintain the shape and the internal space of the sheath 30 in a state wherein the sheath 30 is inserted in a body. Examples of the material applicable here include various resin materials such as polyethylene, polyimides, polyamides, polyester elastomers, and polypropylene.

In accordance with an exemplary embodiment, the sheath 30, for example, can be light-transmitting so that the inside of the sheath 30 is externally visible, which can help ensure, for example, that the conditions of the puncture needle 11 or the implant 9 inserted in the sheath 30 can be visually checked.

In addition, an outer circumferential portion of the sheath 30 can be subjected to a friction-lowering treatment by application of lubricant, for example. The friction-lowering treatment can help permit an operation of inserting the sheath 30 into the connection member 12 and the restriction member 13 to be carried out relatively easily.

As shown in FIG. 8B, the sheath 30 can include the main body 31 to guide a sling 9, and the puncture portion 35 disposed at the distal end of the main body 31 including a position-control portion 38 at the distal end and a dissecting portion 36. The puncture portion 35 having a proximal portion 352.

The puncture portion 35 is configured to rotate in an extended curved line of the main body 31 together with the main body 31. The puncture portion 35 is rotated to create a through hole from the outside the body through the obturator foramens 1102. The main body 31 is hollow, and has a lumen. The sling 9 can be guided through the lumen to be delivered to the desired site. The cross-section of the main body 31 can be a flattened circular shape having generally sharpened ends at the both outer edges of the flattened circle. In accordance with an exemplary embodiment, the distance between inside the both edges defines a larger inner diameter of the lumen. The sharpened ends at the edges of the flattened circle continuously form longitudinal ridges along the longitudinal axis of the main body 31. The ridges can include a first inner ridge A1 (an inner circumferential portion) and a first outer ridge A2 (an outer circumferential portion). In accordance with an exemplary embodiment, the first inner ridge A1 disposed at the inner circumference can have a shorter length than the first outer ridge A2 disposed at the outer circumference. The main body 31 can be made from relatively hard materials, and opposes the collapsing force to be flattened, thereby keeping the lumen patent when inserted in the tissues.

In accordance with an exemplary embodiment, the puncture portion 35 can have a generally flattened cone-shaped member, and has a longitudinal axis in an co-axial or offset extended line of the main body 31. The flattened cone-shaped member can also include a second inner ridge B1 and a second outer ridge B2. The second inner ridge B1 disposed at the inner circumference can have a shorter length than the second outer B2 ridge disposed at the outer circumference. The longitudinal axis is gently curved, and forms an arc along with the extended longitudinal axis of the main body 31. The puncture portion 35 has a generally flattened cone-shaped member including a position-control portion 38 at the distal end, a dissecting portion 36, and the proximal portion 352.

The position-control portion 38 has a rounded distal end protrusion and a tubular member 37 at the distal most end projecting distally along the longitudinal axis of the puncture portion 35 from the flattened cone-shaped member. The tubular member 37 may have a tapered outer shape. The position-control portion 38 controls a position to be dissected in the tissue and determines a puncture route of the puncture portion 35 in the tissue. The dissecting portion 36 is located at the proximal portion of the puncture portion 35 (the flattened cone-shaped member), and can include a generally sharpened ends at the both outer edges in the flattened circle in the cross-section. The dissecting portion 36 has a largest width between both outer edges among the length of the sheath 30 along its longitudinal axis, and broadens the dissection site in the tissue when it dissects the tissues. The dissecting portion 36 has a larger-diameter portion, and a smaller-diameter portion proximally disposed to the larger-diameter portion at the proximal end of the puncture portion 35 (the flattened cone-shaped member). For example, the puncture portion 35 (the flattened cone-shaped member) can have a gradually tapered shape to have a larger diameter from the distal end to the larger-diameter portion around the proximal end, and a smaller diameter at the smaller-diameter portion at the proximal end.

The distal end of the main body 31 can have a smaller outer diameter than that of the larger-diameter portion of the dissection portion 36 of the puncture portion 35. The inner diameter of the dissecting portion 36 can be almost as same as that of the mesh (sling) 9. The puncture portion has further the proximal portion 352. The proximal portion 352 can include a plurality of engaging members (a plurality of legs), that connect the proximal end of the puncture portion 35 and the distal end of the main body 31 by engaging the legs with the inner lumen of the main body 31. The puncture portion 35 can have a co-axial or offset longitudinal axis in an extended line of the main body 31. The position-control portion 38 can have a co-axial or offset longitudinal axis in an extended line of the main body 31.

Support Unit

As shown in FIG. 1, a support unit 17 supporting the puncture member 3 (operating member 7) can be incorporated and disposed in a grasping portion 21 of the frame 2. The support unit 17 can support the puncture member 3 in such a manner that the puncture member 3 can be turned about the shaft portion 73. In addition, the support unit 17 can support the puncture member 3 also in such a manner that the puncture member 3, while being turned about the shaft portion 73, can be moved in a direction orthogonal to the shaft portion 73, for example, in a horizontal direction in a use state (mounted state) of the puncture apparatus 1 in this embodiment.

Insertion Instrument

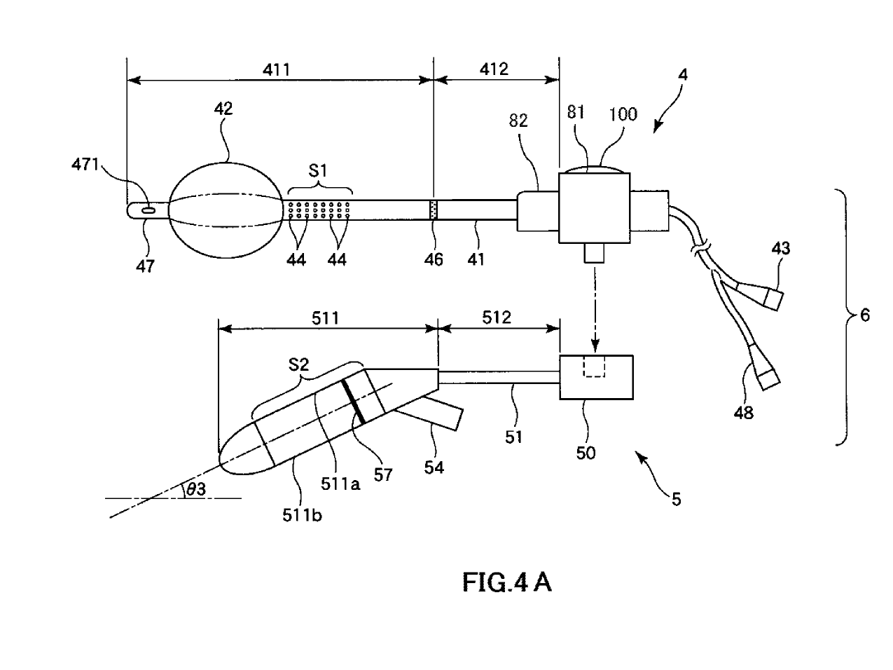

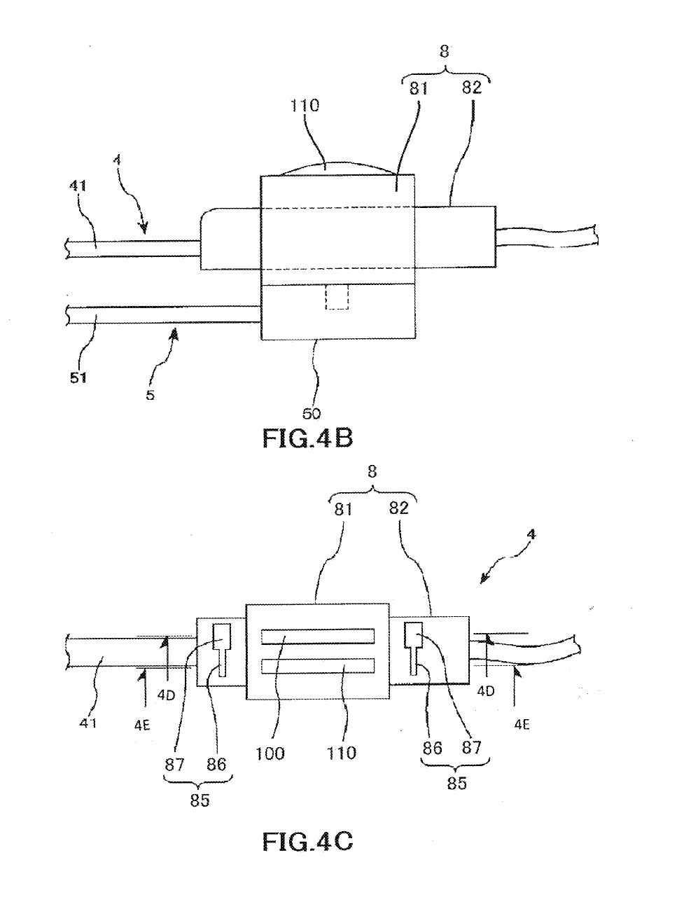

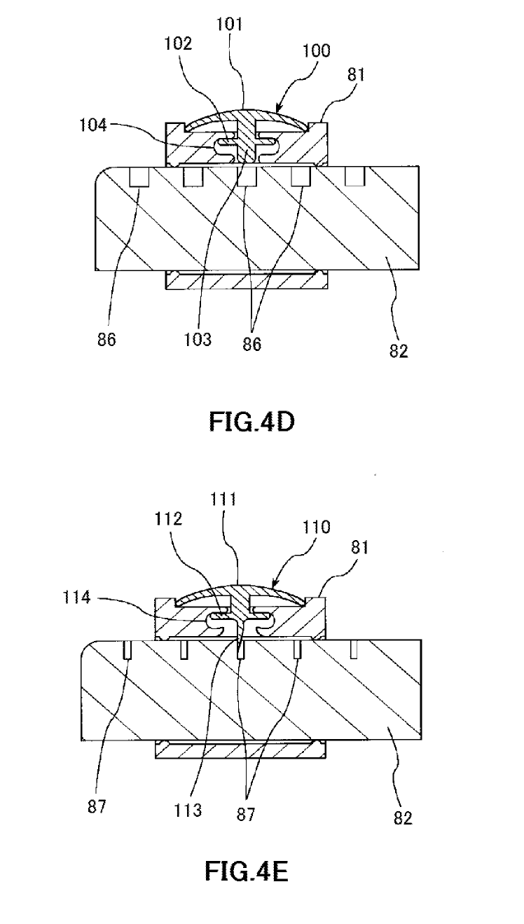

As shown in FIGS. 1 and 4A, the insertion instrument 6 can include a urethral insertion portion (second insertion portion) 41 to be inserted into the urethra 1300, a vaginal insertion portion (first insertion portion) 51 to be inserted into the vagina 1400, and the support part 60 supporting the urethral insertion portion 41 and the vaginal insertion portion 51. As has been mentioned above, the insertion instrument 6 can be composed essentially of the urethral insertion member 4 and the vaginal insertion member 5. The urethral insertion member 4 has the urethral insertion portion 41, and the vaginal insertion member 5 has the vaginal insertion portion 51. In addition, the support part 60 can include a support portion 8, which is possessed by the urethral insertion member 4 and supports the urethral insertion portion 41, and a support portion 50, which is possessed by the vaginal insertion member 5 and supports the vaginal insertion portion 51. In the insertion instrument 6, the urethral insertion member 4 and the vaginal insertion member 51 can be freely detachable by way of the support portions 40 and 50, respectively.

Urethral Insertion Member

The urethral insertion member 4 can include the elongated urethral insertion portion 41 whose portion ranging from a distal end to an intermediate portion of the urethral insertion portion 41 is to be inserted into a urethra 1300, and the support portion 8 which supports the urethral insertion portion 41. Incidentally, in the following, for convenience of description, that portion of the urethral insertion member 4 which is located inside the urethra 1300 (inclusive of a bladder 1310) in the mounted state will be referred to also as “insertion portion 411,” whereas that portion of the urethral insertion member 4 which is exposed via a urethra orifice to the outside of the body in the mounted state and which ranges to the support portion 8 will be referred to also as “non-insertion portion 412.”

The urethral insertion portion 41 is in the shape of a tube with its distal end rounded. In addition, the insertion portion 411 can be provided at its distal portion with an inflatable and deflatable balloon 42 and a urine drain portion 47. In accordance with an exemplary embodiment, the balloon 42 can function as a restriction portion restricting a position in an axial direction of the urethral insertion member 4 in the inside of the urethra 1300. For example, when the puncture apparatus 1 is used, the balloon 42 is inflated after inserted into a patient's bladder 1310. Then, with the balloon 42 caught on a bladder neck, the position of the urethral insertion member 4 relative to the bladder 1310 and the urethra 1300 is fixed. In accordance with an exemplary embodiment, the urine drain portion 47 can be used for draining urine present inside the bladder 1310.

In accordance with an exemplary embodiment, the balloon 42 can extend through the inside of the urethral insertion portion 41, to be connected to a balloon port 43 provided at a proximal portion of the urethral insertion portion 41. A balloon-inflating instrument such as a syringe can be connected to the balloon port 43. When a working fluid (a liquid such as physiological salt solution, or a gas) is supplied from the balloon-inflating instrument into the balloon 42, the balloon 42 is inflated. On the contrary, when the working fluid is drawn out of the balloon 42 by the balloon-inflating instrument, the balloon 42 is deflated. In FIG. 4A, the balloon 42 in its deflated state is drawn in two-dot chain line, whereas the balloon 42 in its inflated state is drawn in solid line.

In accordance with an exemplary embodiment, the urine drain portion 47 can be provided with a drain hole 471, which can provide communication between an inside and an outside of the urine drain portion 47. In addition, the urine drain portion 47 can extend through the inside of the urethral insertion portion 41, to be connected to a urine drain port 48 provided at a proximal portion of the urethral insertion portion 41. Therefore, the urine introduced through the drain hole 471 into the urine drain portion 47 can be drained via the urine drain port 48.

The balloon 42 and the urine drain portion 47 can be configured by use of a double lumen, for example.

In addition, the insertion portion 411 can be formed with a plurality of suction holes 44 at an intermediate portion of the insertion portion 411. The plurality of suction holes 44 can be laid out (or arranged) over a whole range in a circumferential direction of the urethral insertion portion 41. Each of the suction holes 44 can be connected to a suction port 45 provided at a proximal portion of the urethral insertion portion 41, via the inside of the urethral insertion portion 41. A suction device such as a pump can be connected to the suction port 45. When the suction device is operated in a state wherein the urethral insertion portion 41 is inserted in the urethra 1300, a urethral wall can be sucked and fixed onto the urethral insertion portion 41. When the urethral insertion portion 41 is pushed in toward the distal end (toward the inside of the body) under this condition, the urethra 1300 is also pushed in together with the urethral insertion portion 41. As a result, for example, the bladder 1310 can be shifted to such a position as not to overlap with a puncture route for the puncture member 3, whereby the puncture route for the puncture member 3 can be secured. Therefore, puncturing by the puncture member 3 can be carried out relatively accurately and safely. In accordance with an exemplary embodiment, the number of the suction holes 44 is not particularly limited, for example, the number may be one. In addition, a layout of the suction holes 44 is not specifically restricted, for example, the suction holes 44 may be formed in only a part of the range in the circumferential direction of the urethral insertion portion 41.

In addition, at a boundary between the insertion portion 411 and the non-insertion portion 412, a marker 46 can be provided with which to check a depth of insertion of the urethral insertion portion 41 into the urethra 1300. When the urethral insertion portion 41 is inserted in the urethra 1300 and the balloon 42 is located inside the bladder 1310, the marker 46 is located at the urethral orifice, which can permit relatively easy checking of the depth of insertion of the insertion portion 411 into the urethra 1300. In accordance with an exemplary embodiment, the marker 46 is required only to be externally visible, and can be composed essentially of, for example, a colored portion, a recessed and projected portion. For example, a graduation with indications of distance from the distal end of the urethral insertion portion 41 may be provided, in place of the marker 46.

In accordance with an exemplary embodiment, a length of the insertion portion 411 is not particularly limited, and may be set, for example, according to a length of the urethra 1300 and a shape of the bladder 1310 of the patient. The length of the insertion portion 411 can be, for example, about 50 to 100 mm, in view of the fact that the length of a female urethra 1300 is generally about 30 to 50 mm.

A length of the non-insertion portion 412 (a spacing between the urethral orifice and the support portion 8) is not particularly limited. The length can be, for example, not more than about 100 mm, for example, in the range of about 20 to 50 mm. By such a setting, the length of the non-insertion portion 412 can be made appropriate, which can promise relatively enhanced operability. For example, if the length of the non-insertion portion 412 exceeds the above-mentioned upper limit, a center of gravity of the puncture apparatus 1 would, depending on the configuration of the frame 2, be largely deviated from the patient, which can lead to a lowered stability of the puncture apparatus 1 in the mounted state.

In accordance with an exemplary embodiment, the material constituting the urethral insertion member 4 is not specifically restricted. Examples of the material applicable here include various metallic materials such as stainless steels, aluminum, aluminum alloys, titanium, titanium alloys, etc. and various resin materials.

In accordance with an exemplary embodiment, an inclination angle θ2 of the plane f9 relative to a plane f2 orthogonal to an axis J2 of the urethral insertion portion 41 can be, for example, about 20 to 60 degrees, for example, about 30 to 45 degrees, and preferably about 35 to 40 degrees. For example, the main body 31 can be set indwelling in the body so that the angle formed between the plane f9 and a plane orthogonal to the axis of the urethra 1300 can be, for example, about 20 to 60 degrees, for example, about 35 to 45 degrees, and preferably about 30 to 45 degrees. Such a setting can makes the puncturing by the puncture member 3 relatively easily to perform and can shorten the distance of puncture by the puncture member 3.

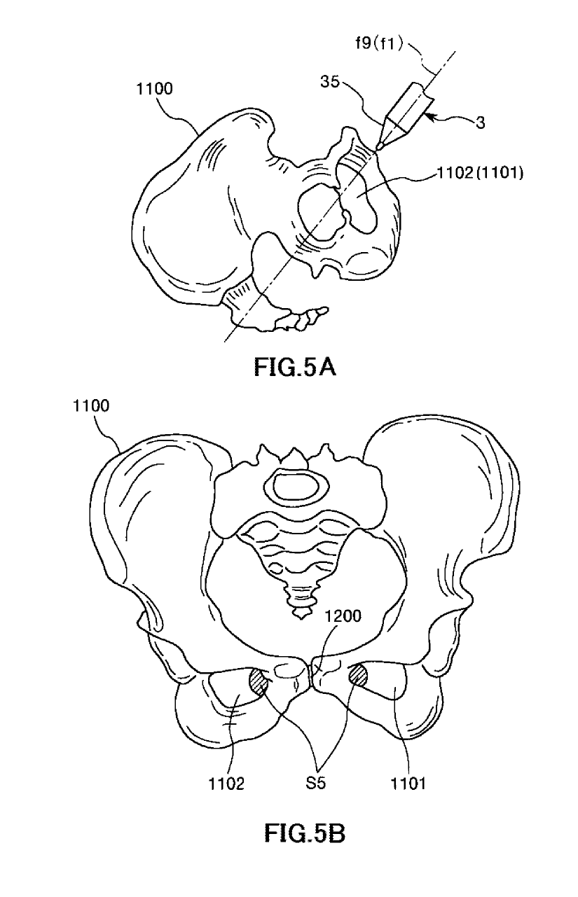

In accordance with an exemplary embodiment, setting the inclination angle θ2 to within the above-mentioned range can help ensure that the puncture member 3 can capture the left and right obturator foramens 1101 and 1102 of the pelvis 1100 wider on a planar basis, so that a wide puncturing space for the puncture member 3 can be secured. For example, in a condition where a patient is put in a predetermined posture (lithotomy position), puncturing by the puncture member 3 can be performed in a direction comparatively nearer to a perpendicular direction relative to the obturator foramens 1101 and 1102. Therefore, the puncturing by the puncture member 3 can be carried out relatively easily. In addition, since the puncturing by the puncture member 3 is performed in a direction comparatively nearer to the perpendicular direction relative to the obturator foramens 1101 and 1102, it can help ensure that the puncture member 3 passes through a shallow part of tissue, so that the puncture member 3 can cross a region between the left and right obturator foramens 1101 and 1102 by passing a short distance. Accordingly, as shown in FIG. 5B, the puncture member 3 can be passed through those regions of the obturator foramens 1101 and 1102 which are near a pubic symphysis 1200, for example, preferably, through safety zones S5. Since safety zones S5 can be regions where there are few nerves and blood vessels the damage to which is to be obviated, puncturing by the puncture member 3 can be performed safely. The result is minimal invasiveness, whereby burden on the patient can be relatively suppressed. Thus, with the inclination angle θ2 set to within the above-mentioned range, puncturing of a patient by the puncture member 3 can be performed more appropriately. In addition, by puncturing at the above-mentioned angle, it is facilitated to aim at a tissue between a middle-part urethra (which refers to an intermediate part in a lengthwise direction of the urethra 1300) and the vagina 1400. The position between the middle-part urethra and the vagina 1400 can be a position suitable as a site where to embed the implant 9 for treatment of urinary incontinence.

In accordance with an exemplary embodiment, in the case where the inclination angle θ2 is less than the aforementioned lower limit or in excess of the aforementioned upper limit, there may arise a problem in that, depending on an individual differences concerning the patient or a posture during the procedure, the puncture member 3 cannot capture the obturator foramens 1101 and 1102 wide on a planar basis or the puncture route cannot be made sufficiently short.

In accordance with an exemplary embodiment, for example, the puncturing is conducted in a condition where either one or both of the urethra 1300 and the vagina 1400 are positionally so shifted as to be pushed toward the inside of the body. Such an operation can permit relatively easy puncturing of the tissue between the middle-part urethra and the vagina 1400. The method pushing in either one of the urethra 1300 and the vagina 1400 toward the inside of the body may be, for example, a method in which the urethral insertion member 4 and/or the vaginal insertion member 5 is inserted into an appropriate position, then, in this condition, the urethra 1300 and/or the vagina 1400 is sucked by the suction holes 44 and 59 (described later) possessed by these members 4 and 5, and thereafter the urethral insertion member 4 and/or the vaginal insertion member 5 is further moved toward the inside of the body along the axis thereof until reaching a predetermined position.

In accordance with an exemplary embodiment, in the condition where at least one of the urethra 1300 and the vagina 1400 has thus been positionally shifted so as to be pushed in toward the inside of the body, the main body 31 of the sheath 30 is made to puncture the living body perpendicularly to the left and right obturator foramens 1101 and 1102 of the pelvis, whereby a passage can be formed in a position suited to implanting of the implant 9.

In accordance with an exemplary embodiment, it can be preferable that a setting is made to cause an orbital path of the main body 31 of the sheath 30 to pass the safety zones S5 of the left and right obturator foramens 1101 and 1102 of the pelvis, at least one of the urethra 1300 and the vagina 1400 is positionally shifted toward the inside of the body so as to locate the orbital path between the middle-part urethra and the vagina 1400, and puncturing is performed along the orbital path of the main body 31, thereby forming the passage.

As shown in FIG. 4D, a locking member 100 is capable of locking the relative movement of the chamber 82 and the hub 81. The locking member 100 has a portion passing through the opening 104 of the hub 81, and has a distal portion 103 capable of being accommodated in a housing 86 of the hub 81. When the distal portion 103 is accommodated in the housing 86, the relative movement of the hub 81 and the chamber 82 is blocked. The support portion 8 can include the hub 81 and the chamber 82, and the locking member 101.

The locking member 100 can have a proximal portion 101 protruding from outside the hub 81. In accordance with an exemplary embodiment, when the proximal portion 101 is pushed (compressed) toward the hub 81, the distal portion 103 is accommodated in the housing 86 (locked by pushing).

As shown in FIG. 4E, the urethra-insertion member 4 can include an attachment member 110 slidably attaching the chamber 82 and the hub 81. The attachment member 110 can includes a switching member (a projection 113) slidably displacing the chamber 82 and the hub 81 along the longitudinal axis when a force is applied to a certain level. The attachment member 110 can have a portion passing through an opening 114 of the hub 81, and can have a projection 113 insertable (engageable) in a recess 87 of the chamber 82. When a force is applied in the direction of the sliding, the projection 113 deforms and displaces by the force so as to disengage the projection 113 from the recess 87, thereby dislocating and reengaging the projection 113 to the adjacent recess 87 longitudinally aligned in the upper surface of the chamber 82.

In accordance with an exemplary embodiment, the attachment member can have a proximal portion protruding from outside the housing. When the proximal portion is pushed (compressed) toward the hub, the locking member can release the accommodated status in the housing (releasing the lock by pushing).

In accordance with an exemplary embodiment, the chamber can have a projecting member (not shown in the drawing) capable of blocking the relative movement of the chamber and the hub on either the chamber or the hub.

Vaginal Insertion Member

As shown in FIGS. 1 and 4A, the vaginal insertion member 5 can include the elongated vaginal insertion portion (first insertion portion) 51 whose portion from a distal end to an intermediate portion of the vaginal insertion portion 51 is inserted into a vagina 1400, and the support portion 50 supporting the vaginal insertion portion 51. For example, in the following, for convenience of description, that portion of the vaginal insertion member 5 which is located in the vagina 1400 in the mounted state will be referred to also as “insertion portion 511,” and that portion of the vaginal insertion member 5 which is exposed via a vaginal orifice to the outside of the living body in the mounted state and which ranges to the support portion 50 will be referred to also as “non-insertion portion 512.”

The insertion portion 511 is elongated. In addition, the insertion portion 511 can extend at an inclination relative to the insertion portion 411 so that the insertion portion 511 is spaced more from the insertion portion 411 on the distal end. With the insertion portion 511 inclined relative to the insertion portion 411, a positional relationship between the insertion portions 411 and 511 can be set closer to the positional relationship between the urethra 1300 and the vagina 1400, as compared with the case where the insertion portion 511 is not inclined in this way. In the mounted state, therefore, the puncture apparatus 1 can be held onto the patient relatively more stably, and the burden on the patient can be relatively mitigated. An inclination angle θ3 of the insertion portion 511 relative to the insertion portion 411 is not particularly limited, for example, the inclination angle θ3 can be, for example, about 0 to 45 degrees, for example, preferably about 0 to 30 degrees, which can enable the above-mentioned effect to be displayed more conspicuously. For example, in the case where the inclination angle θ3 is less than the aforementioned lower limit or in excess of the aforementioned upper limit, there may arise a problem in that, depending on individual differences concerning the patient or the posture during the procedure, the urethra 1300 and/or the vagina 1400 may be deformed unnaturally in the mounted state, which can hamper the puncture apparatus 1 from being stably held.

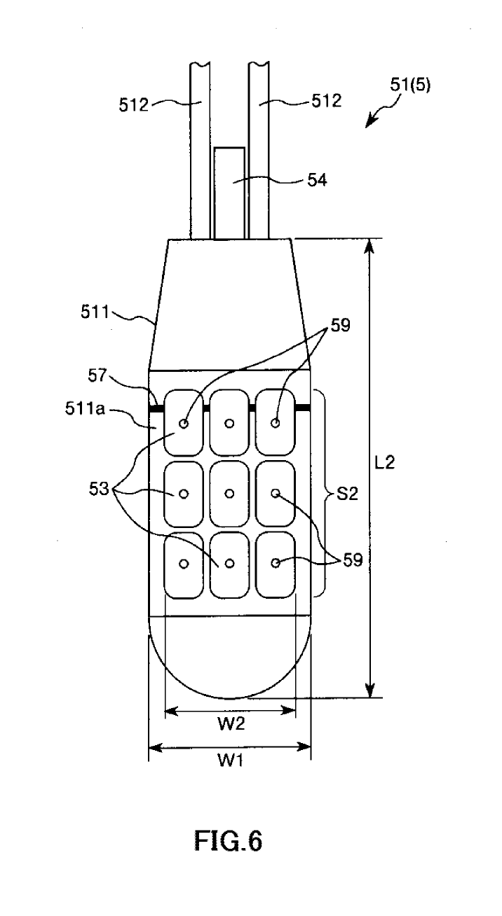

As shown in FIG. 6, the insertion portion 511 is in a flat shape collapsed in a vertical direction of the puncture apparatus 1 (in an array direction of the urethra 1300 and the vagina 1400). In addition, the insertion portion 511 can have a central portion having a substantially constant width and a somewhat rounded distal portion. A length L2 of the insertion portion 511 is not particularly limited, and can be, for example, about 20 to 100 mm, for example, preferably about 30 to 60 mm. A width W1 of the insertion portion 511 is not specifically restricted, and can be, for example, about 10 to 40 mm, for example, preferably about 20 to 30 mm. For example, a thickness of the insertion portion 511 is not particularly limited, and can be, for example, preferably about 5 to 25 mm, for example, preferably about 10 to 20 mm. Set to have such length, width, and thickness, the insertion portion 511 is suited in shape and size to ordinary vaginas. Therefore, stability of the puncture apparatus 1 in the mounted state can be relatively enhanced, and the burden on the patient can be relatively alleviated.

In accordance with an exemplary embodiment, an upper surface (a surface on the urethral insertion portion 41 side) 511a of the insertion portion 511 can be formed with a plurality of bottomed recesses 53. In accordance with an exemplary embodiment, the number of the recesses 53 is not particularly limited, for example, the number may be one. For example, each recess 53 can include a single suction hole 59 in its bottom surface. Each suction hole 59 can be connected to a suction port 54 provided at a proximal portion of the insertion portion 511, through the inside of the insertion portion 511. The suction port 54 can be located in the outside of the living body in the mounted state. A suction device such as a pump can be connected to the suction port 54. When the suction device is operated in the condition where the insertion portion 511 is inserted in the vagina 1400, an anterior wall of vagina, 1410, which is an upper surface of a vaginal wall, is sucked and fixed onto the insertion portion 511. When the vaginal insertion portion 51 with the vaginal wall sucked and fixed thereon is pushed toward the distal end (toward the inside of the body), the vaginal wall can be pushed in together with the vaginal insertion portion 51. Therefore, a configuration and a shape of the vaginal wall can be placed in good order, a puncture route for the puncture member 3 can be secured, and puncturing by the puncture member 3 can be performed relatively accurately and safely.

A region S2 in which the plurality of recesses 53 formed is disposed opposite to a region S1. The dissecting portion 111 of the puncture member 3 passes between these regions S1 and S2. Since the urethra 1300 (which is a lower surface of the urethral wall) is sucked onto the insertion portion 411 in the region S1 and the anterior wall of vagina, 1410, is sucked onto the insertion portion 511 in the region S2, as above-mentioned, the urethral wall and the vaginal wall are spaced wider apart from each other between the regions S1 and S2. By causing the puncture member 3 to pass such a region, therefore, the puncturing by the puncture member 3 can be performed relatively safely.

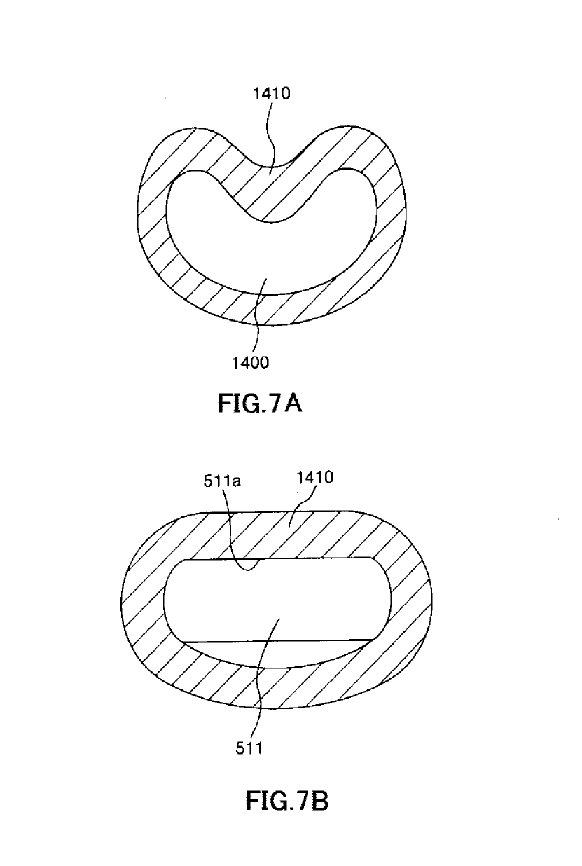

The region S2 ranges over substantially a whole region in a width direction of the upper surface 511a. A width W2 of the region S2 is not particularly limited, and can be, for example, about 9 to 39 mm, for example, preferably about 19 to 29 mm, which can enable the anterior wall of vagina, 1410, to be sucked onto the insertion portion 511 more reliably, without being much influenced by the shape of the vaginal wall. For example, in some patients, the vagina 1400 may be so shaped that part of the anterior wall of vagina, 1410, is hanging down into the inside of the vagina 1400, as shown in FIG. 7A. Even in such a case, setting the width W2 to within the above-mentioned range can help ensure that not only the hanging-down portion but also the portions on both sides of the hanging-down portion can be sucked in an assured manner, as shown in FIG. 7B. Therefore, the anterior wall of vagina, 1410, can be spaced from the urethra 1300 more reliably, without being affected by the shape of the vagina 1400. For example, in this exemplary embodiment, the insertion portion 511 is flat-shaped, so that the anterior wall of vagina, 1410, can be sucked so as to be spaced apart from the urethra 1300. Consequently, the biological tissue between the urethral wall and the vaginal wall can be more expanded (spread).

In addition, the insertion portion 511 can includes a marker (puncture position checking portion) 57 with which a puncture route for the puncture apparatus 1 can be checked. For example, the puncture apparatus 1 can be so fixed as to puncture a region (biological tissue) between the vaginal wall present on an upper side of the position where the marker 57 is located and the urethral wall. Therefore, operability and safety of the insertion instrument 6 can be relatively enhanced. The marker 57 can be provided at least on a lower surface 511b of the insertion portion 511. The lower surface 511b is a surface, which is oriented toward the vaginal orifice, which can be visually confirmed by the operator via the vaginal orifice, in the mounted state. With the marker 57 provided on the lower surface 511b, therefore, the puncture route for the puncture apparatus 1 can be reliably checked. For example, the depth of insertion of the insertion portion 511 into the vagina can also be checked. In accordance with an exemplary embodiment, the marker 57 is required only to be externally visible, and can be configured by use of, for example, a colored portion, or a recessed and projected portion.

The non-insertion portion 512 can be in the shape of a thin bar, which extends substantially in parallel to the urethral insertion portion 41. A spacing D between the non-insertion portion 512 and the urethral insertion portion 41 is not particularly limited, and can be, for example, about 5 to 40 mm, correspondingly to the spacing between the urethral orifice and the vaginal orifice in common women.

In accordance with an exemplary embodiment, a length of the non-insertion portion 512 (a spacing between the vaginal orifice and the support portion 50) is not specifically restricted, and can be, for example, not more than about 100 mm, for example, preferably in the range of about 20 to 50 mm. This permits the non-insertion portion 512 to be appropriate in length, leading to enhanced operability. If the length of the non-insertion portion 512 exceeds the above-mentioned upper limit, the center of gravity of the puncture apparatus 1 would, depending on the configuration of the frame 2, be largely deviated from the patient, which can lead to a lowered stability of the puncture apparatus 1 in the mounted state.

The material constituting the vaginal insertion member 5 is not specifically restricted. Examples of the material applicable here include various metallic materials such as stainless steels, aluminum, aluminum alloys, titanium, titanium alloys, etc. and various resin materials, like the examples of the material for the urethral insertion member 4.

In accordance with an exemplary embodiment, while the urethral insertion member 4 and the vaginal insertion member 5 constituting the insertion instrument 6 can be freely detachable in the puncture apparatus 1, this configuration is not restrictive. For example, a configuration may be adopted in which the urethral insertion member 4 and the vaginal insertion member 5 are non-detachable.

For example, while the members can be fixed to the frame 2 so that the inclination angle θ2 will be constant in the puncture apparatus 1, this configuration is not restrictive. For example, the inclination angle θ2 may be variable, which can permit the inclination angle θ2 to be controlled according to the patient to be treated, which makes the puncture apparatus 1 better in utility.

Implant

As shown in FIG. 13, the implant (biological tissue-supporting indwelling body) 9 is an embeddable (implantable) instrument for treatment of female urinary incontinence, for example, an instrument supporting the urethra 1300. For example, when the urethra 1300 tends to move toward the vaginal wall side, the implant 9 supports the urethra 1300 in such a manner as to restrict its movement in a direction of coming away from the vaginal wall. As the implant 9, for example, a flexible elongate body can be used.

The implant 9 can include an implant main body (band-shaped elongate body) 91, and a bag-shaped wrapping material 92 accommodating the implant main body 91. In addition, the implant main body 91 can include a main body portion 911, and a band 912 interlocked to one end of the main body portion 911. With the implant 9 provided with the wrapping material 92, contamination of the implant main body 91 can be effectively prevented. In accordance with an exemplary embodiment, a guide wire, braid, or string may be used in place of the band 912.

In accordance with an exemplary embodiment, the main body portion 911 can be a reticulated band body. In accordance with an exemplary embodiment, the main body portion 911 may be composed, for example, of a body obtained by intersecting linear members and knitting them into a reticulated form, that is, a reticulated braiding. Examples of the linear member include those, which are circular in cross-sectional shape, those that are flat-shaped in cross section, etc.

The materials constituting the main body portion 911, the band 912 and the wrapping material 92 are each not specifically restricted. Examples of the materials applicable here include various biocompatible resin materials such as polypropylene, polyesters, or nylon, and fibers, etc.

In accordance with an exemplary embodiment, the implant 9 is not restricted to the above-mentioned reticulated body, insofar as it can produce an effect equivalent to the aforementioned.

Method of Using the Puncture Apparatus

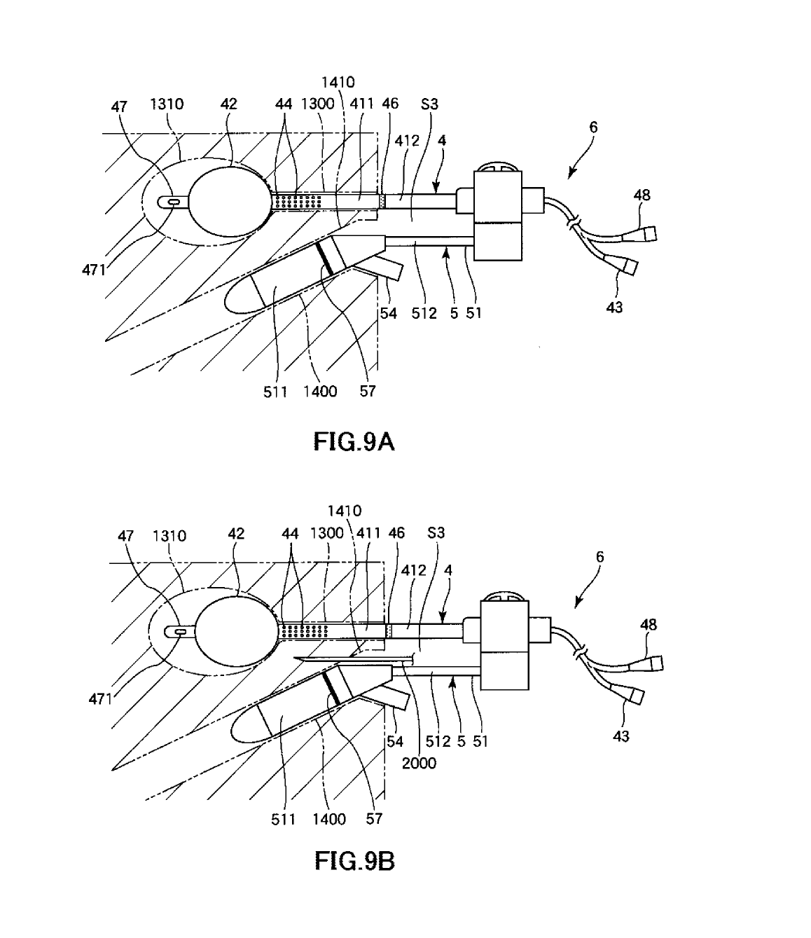

Now, a method of using the puncture apparatus 1 (operation procedure) will be described below referring to FIGS. 9A to 11. Prior to placement of the implant 9 between the urethra 1300 and the vagina 1400, by use of the puncture apparatus 1 (by the puncturing method), a bow-like puncture hole can be formed by placing the implant 9 indwelling in a biological tissue.

In accordance with an exemplary embodiment, first, a patient is put in a lithotomy position on an operating table, and the insertion instrument 6 is mounted onto the patient, as shown in FIG. 9A. For example, first, the urethral insertion portion 41 of the urethral insertion member 4 can be inserted into the patient's urethra 1300. In this instance, while checking the depth of insertion by observing the marker 46, the balloon 42 is disposed inside the bladder 1310. The urethra 1300 can be rectified into a predetermined shape by the urethral insertion portion 41 having the predetermined shape. In the case of this embodiment, the urethra 1300 can be rectified into a rectilinear shape by the urethral insertion portion 41 having the rectilinear shape.

Next, the balloon 42 is inflated, and, if necessary, urine is drained from the inside of the bladder 1310 through the drain hole 471. Then the operator measures a length of the urethra 1300 in a subject, changes the length of the urethra-insertion member 4 so as to form a through hole passing the living tissue under the mid-urethra adjacent the center of the length of the urethra 1300, and connecting a puncture member 3 to the urethra-insertion member 4 after changing the length of the urethra-insertion member 4.

In addition, the vaginal insertion portion 51 of the vaginal insertion member 5 is inserted into the patient's vagina 1400. In this instance, while checking the puncture position by observing the marker 57, the vaginal insertion portion 51 is inserted to an appropriate depth. Then the urethral insertion member is connected to the vaginal insertion member to fixing the support portions 40 and 50. By this, mounting of the insertion instrument 6 onto the patient is completed. In this state, the non-insertion portions 412 and 512 can be spaced apart from each other, the support part 60 is spaced from the body surface between the urethral orifice and the vaginal orifice, and that body surface is exposed. In addition, in the case where the insertion portion 511 and the anterior wall of vagina, 1410, are spaced apart from each other and a gap (space) is formed therebetween, a space S3 causing a syringe to puncture from the body surface between the urethral orifice and the vaginal orifice to a biological tissue between the urethra and the vagina is formed.

Subsequently, suction devices are connected to the suction ports 45 and 54, and the suction devices are operated, whereby the urethra 1300 is sucked onto the urethral insertion portion 41, and the anterior wall of vagina, 1410, is sucked onto the vaginal insertion portion 51. For example, when the urethra 1300 is properly sucked onto the urethral insertion portion 41, the suction holes 44 are closed with the urethral wall, so that the suction via the suction port 45 is stopped or weakened. Similarly, when the anterior wall of vagina, 1410, is properly sucked onto the vaginal insertion portion 51, the suction holes 59 are closed with the vaginal wall, so that the suction via the suction port 54 is stopped or weakened. Therefore, based on the conditions of suction via the suction ports 45 and 54 (for example, the magnitude of sounds generated attendant on the suction via the suction ports 45 and 54), the operator can check whether or not the urethra 1300 and the anterior wall of vagina, 1410, have been properly sucked onto the urethral insertion portion 41 and the vaginal insertion portion 51, respectively. In accordance with an exemplary embodiment, the insertion instrument 6 may be provided with a checking mechanism for mechanical checking of the sucked state. The checking mechanism is not specifically restricted, insofar as it enables checking of the sucked state. For example, the checking mechanism may be configured to include a flow measurement unit (negative-pressure sensor) measuring a flow rate through the suction port 54 and a decision unit deciding whether or not the sucking is being properly done, based on the measurement results sent from the flow measurement unit.

Next, humoral dissection is performed. Specifically, as shown in FIG. 9B, the puncture needle of a syringe 2000 is caused to puncture the anterior wall of vagina, 1410, by way of the space (space S3) between the insertion portion 511 and the anterior wall of vagina, 1410, and a liquid such as physiological salt solution or local anesthetic is injected into the biological tissue between the urethra 1300 and the vagina 1400 (between the regions 51 and S2). As a result, the biological tissue between the regions 51 and S2 is expanded, the urethra 1300 is pressed against the urethral insertion portion 41, and the anterior wall of vagina, 1410, is pressed against the vaginal insertion portion 51.

In accordance with an exemplary embodiment, the suction through the suction holes 44 and 59 can be continuously conducted also during the humoral dissection. When the urethra 1300 is pressed against the urethral insertion portion 41 due to the humoral dissection, the urethra 1300 is further sucked onto the urethral insertion portion 41, so that the suction via the suction port 45 is stopped or weakened. Similarly, when the anterior wall of vagina, 1410, is pressed against the vaginal insertion portion 51, the anterior wall of vagina, 1410, is further sucked onto the vaginal insertion portion 51, so that the suction via the suction port 54 is stopped or weakened. Based on the conditions of suction via the suction ports 45 and 54, therefore, the operator can check whether or not the humoral dissection has been properly performed.

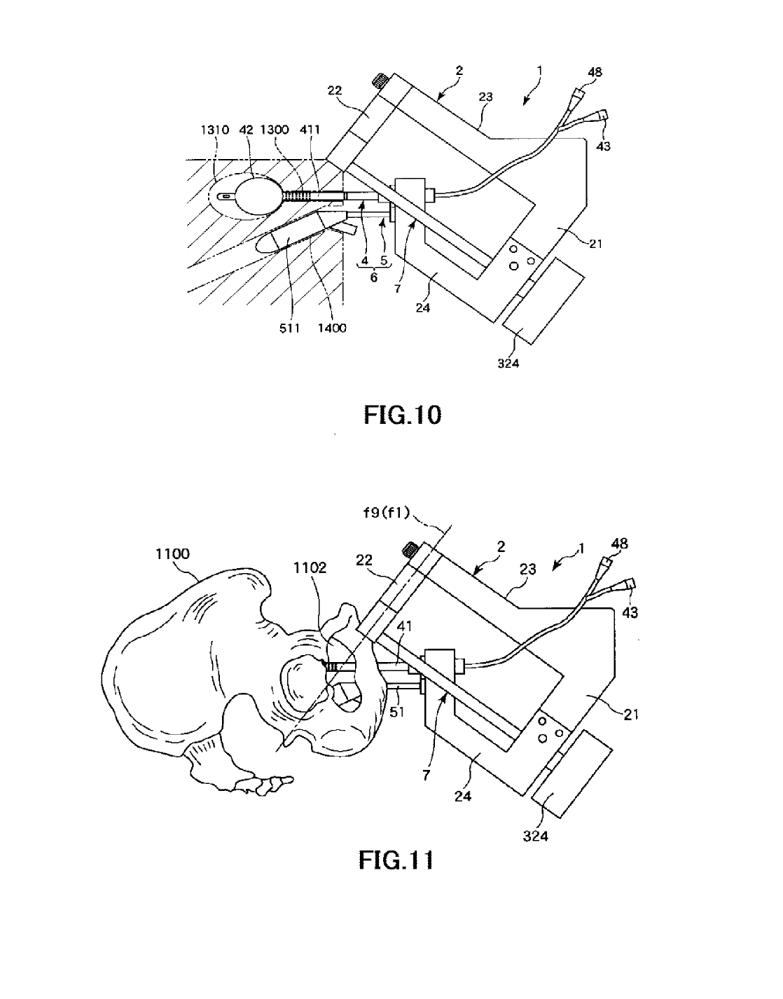

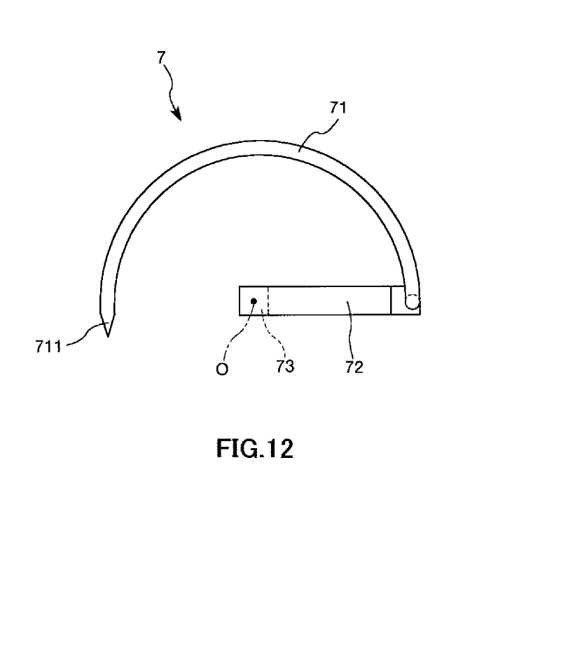

After the humoral dissection is conducted so that the urethra 1300 and the anterior wall of vagina, 1410, are thereby sufficiently spaced apart from each other, the puncture apparatus 1 is set indwelling on the body surface H at a right-hand inguinal region of the patient or at a region near the inguinal region. Then, as shown in FIG. 10, the insertion instrument 6 can be fixed to the frame 2, which results in that the puncture apparatus 1 is mounted on the patient. In this state, a positional relationship between the pelvis 1100 and the puncture apparatus 1 is as shown in FIG. 11.

Method of Using the Puncture Apparatus

As shown in FIG. 14, the puncture apparatus 1 is set into the mounted state, and the puncture portion 35 is set in abutment on a body surface H at the right-hand inguinal region of a patient or a region in the vicinity thereof (first region).

Next, while grasping an interlock portion 23 of the frame 2 by one hand, for example, an interlock portion 72 of the operating member 7 is grasped by the other hand, and the operating member 7 is rotated clockwise. This can help ensure that the puncture portion 35 of the puncture member 3 punctures the body surface H at the right-hand inguinal region of the patient or the region in the vicinity thereof (first region) by the puncture, to enter the body.

Then, when a turning angle of the operating member 7 as measured from the state shown reaches 90 degrees, the puncture portion 35 crosses a region between the urethra 1300 and the vagina 1400.

When the turning angle of the operating member 7 as measured from the state reaches 180 degrees, the dissecting portion 36 has passed the obturator foramen 1102 and reaches a position immediately beneath the body surface H at the left-hand inguinal region or a region in the vicinity thereof.

By the puncturing operation as above, a puncture hole in a bow-like shape can be formed proximate to the target puncture route R in the living body. This puncture needle also is in the state of reliably passing between the urethra 1300 and the vagina 1400, like that in the first embodiment described above. Thus, the puncture apparatus 1 in this embodiment also can help ensure that puncturing of a biological tissue can be carried out relatively accurately.

Next, the puncture portion 35 is detached from the sheath 30, and the operating member 7 is rotated in a direction reverse to the aforementioned. This results in that the inserted portion 71 of the operating member 7 is pulled out of the sheath 30, with the sheath 30 left in the living body.

Subsequently, the implant main body 91 is inserted into the sheath 30 while being taken out of a wrapping material 92, and a band 912 is set into a state of protruding from the proximal end opening and the distal end opening of the sheath 30.

Next, the sheath 30 can be split into the distal split piece 32 and the proximal split piece 33.

Subsequently, the connection between the distal split piece 32 and the proximal split piece 33 is canceled, then the distal split piece 32 is pulled distally out of the living body, and the proximal split piece 33 is pulled proximally out of the living body. In this case, the distal split piece 32 and the proximal split piece 33 moved in the opposite directions substantially simultaneously. For example, the distal split piece 32 and the proximal split piece 33 can be moved along an arcuate path such as to follow their own shape, which can help permit the sheath 30 to be smoothly removed from the living body. As the distal split piece 32 and the proximal split piece 33 are gradually taken out of the living body as above-mentioned, the surrounding tissue having been forced open (expanded) by the sheath 30 returns into an original position, and the tissue comes into contact with the implant main body 91, gradually from a central portion toward both end portions of the implant main body 91. As above-mentioned, the distal split piece 32 and the proximal split piece 33 are moved in directions along their shapes, and the sheath 30 has an internal space such that the implant main body 91 can be moved therein under a sufficiently low friction, which can help ensure that the implant main body 91 can be placed indwelling in an as-is state, with no unnecessary tensile force exerted thereon. Accordingly, there is no need for control of tension on the implant main body 91. As a result of these operations, the implant main body 91 is in the state of being embedded in the living body.

In addition, with the sheath 30 thus split into the split pieces and removed from the living body as the split pieces, pulling-out of the sheath 30 from the living body can be carried out easily. For example, this pulling-out method can help ensure that the split pieces 32 and 33 being pulled out exert little influence on a posture of the main body portion 911 in the region between the urethra 1300 and the vagina 1400.

In addition, the puncture apparatus and puncturing method according to the present disclosure may be a combination of arbitrary two or more of the configurations (characteristic features) adopted in the aforementioned embodiments.

For example, while the puncture portion is detachably held on the main body of the sheath in the fifth embodiment above, this configuration is not restrictive. For instance, a configuration in which the puncture portion is fixed to the main body, such as a configuration wherein the main body and the puncture portion are integrally formed, may also be adopted. In that case, after a living body is punctured by the puncture member and the puncture portion is made to protrude to the outside of the living body, the puncture portion may be cut by use of, for example, a pair of scissors, whereby the distal end opening of the main body can be opened.

While a configuration in which the main body of the sheath can be separated into a distal split piece and a proximal split piece has been described in the fifth embodiment above, this main body configuration is not restrictive. A configuration in which a distal end portion and a proximal end portion of the main body are not separable from each other may also be adopted. Specifically, the main body may be configured to be a single tube. In that case, the aforementioned state-maintaining mechanism is also omitted.

In addition, while a configuration in which the implant main body is inserted into the puncture member after the puncture member is placed in the inside of a living body has been described in the above embodiments, this configuration is not restrictive. The implant main body may be accommodated in the puncture member, from the beginning. In that case, it is preferable, for example, for the implant main body to be set fixed relative to the dissecting portion.

For example, while the case where the puncture apparatus of the present disclosure is applied to an apparatus to be used in embedding in a living body an embeddable implant for treatment of female urinary incontinence has been described in the above embodiments, this is not restrictive of the use of the puncture apparatus.

Examples of the object to which the present disclosure is applicable include pelvic floor diseases inclusive of excretory disorders (urinary urgency, frequent urination, urinary incontinence, fecal incontinence, urinary retention, dysuria, etc.), pelvic organ prolapse, vesicovaginal fistula, urethrovaginal fistula, and pelvic pain, which would be attendant on weakening of the group of pelvic floor muscles. The pelvic organ prolapse include such diseases as cystocele, enterocele, rectocele, and hysterocele, or such diseases as anterior vaginal prolapse, posterior vaginal prolapse, vaginal apical prolapse, and vaginal vault prolapse, which are denominations based on classification of the vaginal wall part being prolapsed.

In addition, examples of overactive tissue include the bladder, vagina, uterus, and bowels. On the other hand, examples of lessactive tissue include bones, muscles, fascias, and ligaments. Especially in relation to the pelvic floor diseases, examples of the lessactive tissue include obturator fascia, coccygeus fascia, cardinal ligament, uterosacral ligament, and sacrospinous ligament.

Examples of the procedure interlocking an overactive tissue in the pelvic floor disorder with the lessactive tissue, there are included a retropubi sling surgery, a transobturator sling surgery (Transobturator Sling Surgery; Transobturator Tape: TOT), a tension-free vaginal mesh (Tension-free Vaginal Mesh: TVM) surgery, a uterosacral ligament suspension (Uterosacral Ligament Suspension: USLS) surgery, an iliococcygeus fascia fixation surgery, and a coccygeus fascia fixation surgery.

It should be understood by those skilled in the art that various modifications, combinations, sub-combinations and alterations may occur depending on design requirements and other factors insofar as they are within the scope of the appended claims or the equivalents thereof.

What is claimed is:

1. A puncture assembly comprising:
a main body to guide a sling; and
a puncture portion disposed at a distal end of the main body, the puncture portion including a position-control portion at the distal end of the main body and a dissecting portion.

2. The puncture assembly of claim 1, wherein the puncture portion is configured to rotate in an extended curved line of the main body together with the main body to create a through hole from an outside a living body through obturator foramens of the living body.

3. The puncture assembly of claim 1, wherein the main body is hollow and has a lumen; and
the sling is guided through the lumen to be delivered to a desired site.

4. The puncture assembly of claim 1, wherein a cross-section of the main body is a flattened circular shape having generally sharpened ends at both outer edges of the flattened circle, and wherein the sharpened ends at the edges of the flattened circle continuously form longitudinal ridges along the longitudinal axis of the main body.

5. The puncture assembly of claim 4, wherein the longitudinal ridges comprise:
a first inner ridge and a first outer ridge, wherein the first inner ridge is disposed at an inner circumference having a shorter length than the first outer ridge disposed at an outer circumference.

6. The puncture assembly of claim 1, wherein the main body comprises:
a distal split piece and a proximal split piece, and wherein the distal split piece and the proximal split piece are approximately equal in length.

7. The puncture assembly of claim 1, wherein the puncture portion comprises:
a flattened cone-shaped member, and has a longitudinal axis in a co-axial or offset extended line of the main body.

8. The puncture assembly of claim 7, wherein the flattened cone-shaped member comprises:
a second inner ridge and a second outer ridge, the second inner ridge disposed at the inner circumference having a shorter length than the second outer ridge disposed at the outer circumference.

9. The puncture assembly of claim 7, wherein the longitudinal axis of the puncture portion is curved, and forms an arc along with the extended longitudinal axis of the main body.

10. The puncture assembly of claim 1, wherein the position-control portion has a rounded distal end protrusion and a tubular member at the distal end projecting distally along the longitudinal axis of the puncture portion from the flattened cone-shaped member, and the tubular member has a tapered outer shape.

11. A puncture assembly comprising:
a sheath, the sheath including a main body to guide a sling, and a puncture portion disposed at a distal end of the main body, the puncture portion including a position-control portion at the distal end of the main body and a dissecting portion, wherein the puncture portion is configured to rotate in an extended curved line of the main body together with the main body to create a through hole from an outside a living body through obturator foramens of the living body; and
an insertion portion configured to be inserted into a hollow lumen of the sheath, the insertion portion having an arcuate shape corresponding to a shape of the sheath.

12. The puncture assembly of claim 11, wherein a distal portion of the insertion portion is tapered, and
the puncture portion has a proximal portion, the proximal portion including a plurality of engaging members that connect a proximal end of the puncture portion and a distal end of the main body by engaging the plurality of engaging members with the lumen of the main body.

13. The puncture assembly of claim 11, wherein a cross-section of the main body is a flattened circular shape having generally sharpened ends at both outer edges of the flattened circle, and wherein the sharpened ends at the edges of the flattened circle continuously form longitudinal ridges along the longitudinal axis of the main body; and
the longitudinal ridges include a first inner ridge and a first outer ridge, wherein the first inner ridge is disposed at an inner circumference having a shorter length than the first outer ridge disposed at an outer circumference.

14. The puncture assembly of claim 11, wherein the main body comprises:
a distal split piece and a proximal split piece, and wherein the distal split piece and the proximal split piece approximately equal in length.

15. The puncture assembly of claim 11, wherein the puncture portion comprises:
a flattened cone-shaped member, and has a longitudinal axis in an co-axial or offset extended line of the main body;
the flattened cone-shaped member includes a second inner ridge and a second outer ridge, the second inner ridge disposed at the inner circumference having a shorter length than the second outer ridge disposed at the outer circumference; and
wherein the longitudinal axis of the puncture portion is curved, and forms an arc along with the extended longitudinal axis of the main body.

16. The puncture assembly of claim 11, wherein the position-control portion has a rounded distal end protrusion and a tubular member at the distal end projecting distally along the longitudinal axis of the puncture portion from the flattened cone-shaped member, and the tubular member has a tapered outer shape.

17. A method of forming a path in living body tissue, the method comprising:
inserting an insertion member into a living body, the insertion member comprising at least one of a urethral-insertion portion that is inserted into a urethra and has an elongated shape and a vaginal-insertion portion that is inserted into a vagina and has an elongated shape;
inserting a puncture assembly into the living body, the puncture assembly comprising a sheath having a main body to guide a sling, and a puncture portion disposed at a distal end of the main body, the puncture portion including a position-control portion at the distal end of the main body and a dissecting portion, wherein the puncture portion is configured to rotate in an extended curved line of the main body together with the main body to create a through hole in a biological tissue from an outside a living body through obturator foramens of the living body; and
detecting a positional relationship of the distal end of the main body of the puncture assembly with the position-control portion of the sheath; and
determining a puncture route of the puncture portion of the puncture assembly.

18. The method of claim 17, comprising:
inserting an insertion portion into a hollow lumen of the sheath, the insertion portion having an arcuate shape corresponding to a shape of the sheath; and
attaching the insertion portion to an operating member, which is configured to rotate the puncture assembly in a rotational direction about a rotation center while the insertion member remains inserted in the living body to move the puncture assembly along a path of rotational movement.

19. The method of claim 18, comprising:
rotating the puncture assembly in the rotational direction about the rotation center while the insertion member remains inserted in the living body to move the puncture assembly along the path of rotational movement, the portion of the living body into which the insertion member is inserted being located relative to the rotation center of the path of rotational movement of the puncture assembly such that a near-side of the insertion member is the side of the insertion member located closer to the rotation center, and a far-side of the insertion member is the side of the insertion member located farther from the rotation center; and
continuing to rotate the puncture assembly in the rotational direction about the rotation center while the insertion member remains inserted in the living body to cause the puncture portion to puncture a surface of the living body tissue.

20. The method of claim 19, wherein the main body comprises a distal split piece and a proximal split piece, and wherein the distal split piece and the proximal split piece approximately equal in length;
inserting an implant into the lumen of the main body with the main body in the living body; and
removing the main body of the puncture assembly by:
pulling the distal split piece distally out of the living body; and
pulling the proximal split piece proximally out of the living body.
